(19) United States
(12) Patent Application Publication
(10) Pub. No.: US 2020/0323678 A1
(43) Pub. Date: Oct. 15, 2020

(54) DENTAL MOUTH GUARD TO PREVENT BRUXISM

(71) Applicants: James S. Fallon, Laguna Niguel, CA (US); Richard Jung, Laguna Niguel, CA (US)

(72) Inventors: James S. Fallon, Laguna Niguel, CA (US); Richard Jung, Laguna Niguel, CA (US)

(21) Appl. No.: 16/382,683

(22) Filed: Apr. 12, 2019

(51) Int. Cl. A61F 5/56; A61C 9/00

(52) U.S. Cl. CPC A61F 5/566; A61F 2005/563; A61C 9/0006

(57) ABSTRACT

A oral appliance (i.e., a dental mouth guard) configured so as to fit in the mouth and over at least one (e.g., the upper) set of teeth of a user during sleep to prevent bruxism and the grinding together of the user's upper and lower sets of teeth. The mouth guard has a relatively soft heat responsive and arcuate shaped upper impression tray that is molded over a relatively hard and arcuate shaped lower chassis. The mouth guard is removably received within and transported by a fitting tray to be submerged in boiling water. After it is heated, the fitting tray relocates the mouth guard to the user's mouth where a biting force is applied by the user to the upper impression tray so that an accurate and custom impression of the user's teeth is made therewithin. The mouth guard is now removed from the fitting tray which is then discarded.

### BACKGROUND OF THE INVENTION

### 1. Field of the Invention

This invention relates to an oral dental appliance (i.e., a mouth guard) that is configured to fit in the mouth and over the teeth of a user during sleep to prevent bruxism and the grinding together of the user's upper and lower sets of teeth. The mouth guard is transported by a disposable fitting tray by which the mouth guard is heated so that an accurate impression of the user's teeth can be made therein in response to a biting force generated by the user.

### 2. Background Art

Bruxism is known to occur during sleep when an individual unconsciously grinds his upper and lower sets of teeth together. As a consequence, the individual's teeth can ultimately wear down resulting in permanent damage to the teeth and pain to the individual. Therefore, an inexpensive and reliable means is desirable to enable the individual to avoid bruxism throughout the night without involving the intervention of a health care worker. In particular, the availability of a reusable mouth guard that could fit in the mouth and over at least one set of the individual's teeth would be advantageous for treating bruxism.

As an important consideration, any such mouth guard should preferably be manufactured from an impressionable material and thereby have the capability of being shaped by the individual to create an accurate custom fit over and against his teeth. In this manner, the mouth guard will be less likely to shift relative to the sleeping individual's teeth and not fall out of his mouth which would render the device useless. In this same regard, customizing the mouth guard must be accomplished without subjecting the individual to harm such as, for example, by burning himself while heating and softening the impressionable mouth guard. Likewise, it would also be preferable that the mouth guard need not be carried in the hand of the individual while being transported between a source of heat and his mouth during the customization process so that the mouth guard will not be accidentally dropped into the source of heat.

One example of a combination oral appliance that is adapted to fit in the mouth and over the teeth of a sleeping individual and a detachable impression tray with an integral handle by which the oral appliance can be transported from a source of heat to the mouth of the individual so that the appliance can be heated and customized to the individual's teeth is available by referring to U.S. patent application Ser. No. 15/948,124 filed May 18, 2018. Once the oral appliance has been customized to fit the teeth of the individual, the impression tray is separated therefrom and discarded. The oral appliance has a set of open cells formed in a copolymer material by which to enable a more accurate customization of the appliance with respect to the individual's teeth.

### SUMMARY OF THE INVENTION

In general terms, disclosed herein is an oral appliance (i.e., a dental mouth guard) that is adapted to be customized to fit in the mouth and over either one of the upper or lower arched sets of teeth of a user during sleep to prevent bruxism and the grinding together of the user's upper and lower sets of teeth. The dental mouth guard has a heat responsive and arcuate shaped upper impression tray that is ideally manufactured from a soft polymer material. The upper impression tray is molded over an arcuate shaped lower chassis that is ideally manufactured from a polymer material that is harder and denser than the material from which the upper impression tray is manufactured. The polymer material of the arcuate upper impression tray has a set of open cells (i.e., holes) formed therein and extending therearound so that the polymer material all flow around the teeth when heated to create a reliable custom fit. A protective bite-resistant pad is formed in and extends continuously around the arcuate lower chassis to lie below the holes formed in the upper impression tray. A buffer wall stands upwardly from the front of the lower chassis. The buffer wall is embedded in the front of the upper impression tray to thereby establish a smooth protective surface which prevents the formation of bumps around the upper impression tray that could irritate the user's lip when the mouth guard is worn at night.

The dental mouth guard is removably received within and transported by a disposable fitting tray. The fitting tray has an arcuate channel to accommodate the mouth guard therewithin and a handle attached to and extending from the channel. A pair of adjustment slots are formed in the channel to permit the channel to flex depending upon the size of the user's jaw and the biting force generated thereby. The handle is grasped and the mouth guard is carried within the fitting tray to a source of heat (e.g., a pot of boiling water) into which the guard is submerged and heated. The heated mouth guard is removed from the source of heat, permitted to momentarily cool, and placed into the user's mouth. The user then bites down on the softened upper impression tray of the mouth guard to create an accurate impression of his (upper) set of teeth. Next, the mouth guard is removed from the user's mouth and transported by the fitting tray to a source of cold water where the mouth guard is cooled to cause the impression of his teeth to be set. The mouth guard is now separated from the fitting tray so as to be ready for use at which time the fitting tray is discarded.

### BRIEF DESCRIPTION OF THE DRAWINGS

FIG. 1 is a perspective view of a dental mouth guard according to a preferred embodiment of this invention configured to fit in the mouth and over the teeth of an individual to prevent bruxism;

FIG. 2 is a rear exploded view of the dental mouth guard shown in FIG. 1;

FIG. 3 is a front exploded view of the dental mouth guard shown in FIG. 1;

FIG. 4 is a bottom exploded view of the dental mouth guard shown in FIG. 1;

FIG. 5 is a cross-section of the dental mouth guard taken along lines 5-5 of FIG. 2;

FIG. 6 is a cross-section of the dental mouth guard shown in FIG. 2 following its one-piece assembly;

FIG. 7 shows the dental mouth guard in combination with a disposable fitting tray to be used during the steps of forming an accurate impression of the user's (upper) set of teeth in the mouth guard prior to its first use; and

FIG. 8 shows the dental mouth guard separated from the disposable fitting tray and positioned over the upper set of teeth of the individual during sleep.

### DESCRIPTION OF THE PREFERRED EMBODIMENT

Referring to the drawings, details are now provided of an oral appliance (i.e., a dental mouth guard) 1 that is configured and has particular application to fit within the mouth and be positioned over one of the upper or lower arched sets of teeth of a user during sleep in order to reduce bruxism and the unconscious grinding together of the user's upper and lower sets of teeth during sleep. As will be explained in greater detail hereinafter, the dental mouth guard 1 of this invention can be customized by the user to closely conform to the shape of the set of teeth over which the mouth guard will be worn during sleep. In this manner, the mouth guard 1 will be less likely to move off the teeth and perhaps fall out of the user's mouth as he tosses and turns and opens and closes his mouth throughout the night.

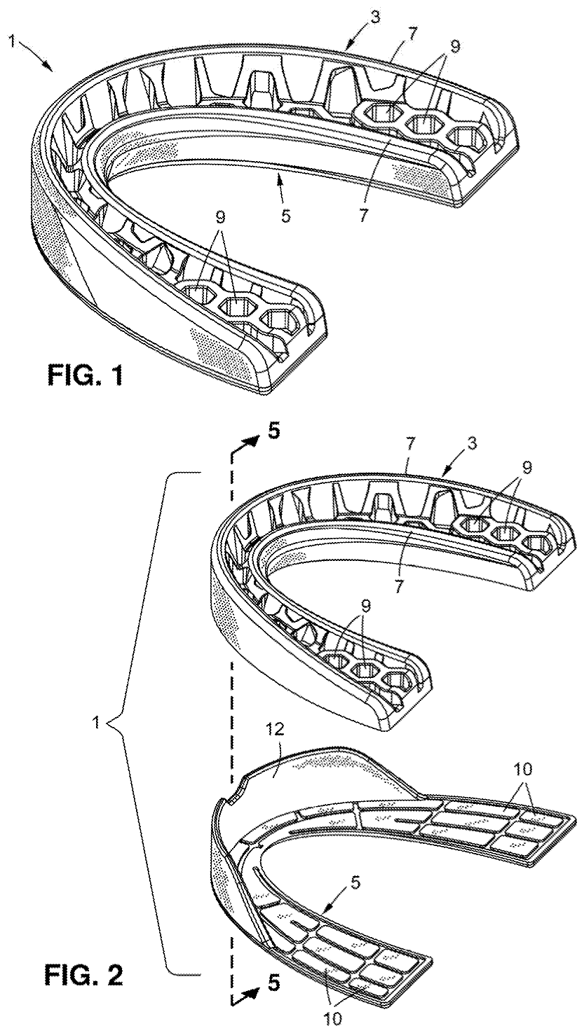

As is best shown in FIGS. 1-4, the dental mouth guard 1 includes an upper impression tray 3 and a lower chassis 5 that are molded together one above the other. Each of the upper impression tray 3 and the lower chassis 5 is arcuately (i.e., arch) shaped so that the mouth guard, once assembled and made ready for use, will lay flush against one set of the user's teeth. The upper impression tray 3 of the mouth guard 1 is preferably manufactured from a relatively soft and impressionable copolymer material such as that known commercially as Elvax 150. The lower chassis 5 is manufactured from a relatively hard copolymer material such as that known commercially as Elvax 750. The material from which the lower chassis 5 is manufactured is both harder and denser than the material from which the upper impression tray 3 is manufactured.

An outer bite impression channel 7 which extends around the arcuate upper impression tray 3 of the mouth guard 1 surrounds a set of open cells or holes 9 that are molded into the soft impressionable material from which the impression tray is manufactured. By way of example only, the set of holes 9 extend around the impression channel 7 in a honeycomb pattern with each hole having a hexagonal shape. The holes 9 are uniformly spaced from one another and run continuously around the outer bite impression channel 7 of the upper impression tray 3. It is however to be understood that the shape and number of the holes 9 that are molded into the upper impression tray 3 are a matter of choice and should not to be considered a limitation of this invention.

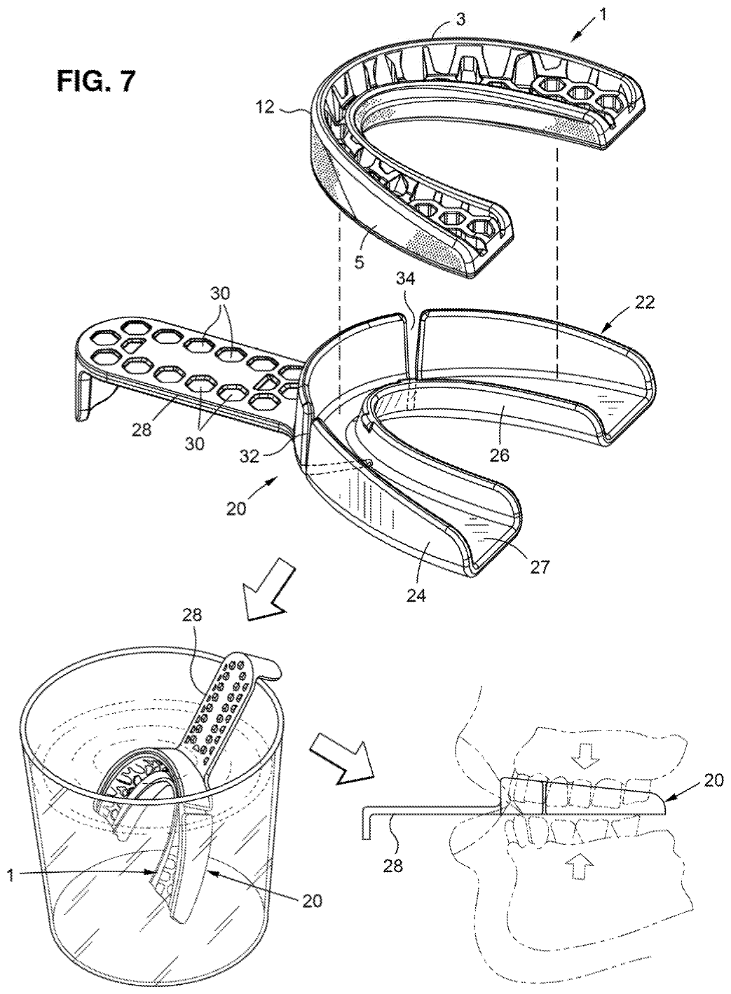

The set of holes 9 that are molded into the upper impression tray 3 of the dental mouth guard 1 advantageously enable a custom fit of the mouth guard 1 so as to lie in surrounding engagement with one of the user's upper or lower sets of teeth when the mouth guard is initially prepared for use. That is, and as will be explained while referring to FIG. 7, the upper impression tray 3 and the lower chassis 5 of the mouth guard 1 are first heated and then placed into the user's mouth so that an impression of one (e.g., the upper) set of teeth will be made when the user bites down into the relatively soft and less dense upper impression tray 3 to cause the tray 3 to be compressed against the relatively hard and more dense lower chassis 5. By virtue of the set of holes 9 that are molded into the upper impression tray 3, the heated soft material of the upper impression tray 3 will be deformed and flow substantially evenly over the edges of and the gaps between the user's (e.g., upper) set of teeth whereby to make an accurate and close fitting impression of his teeth.

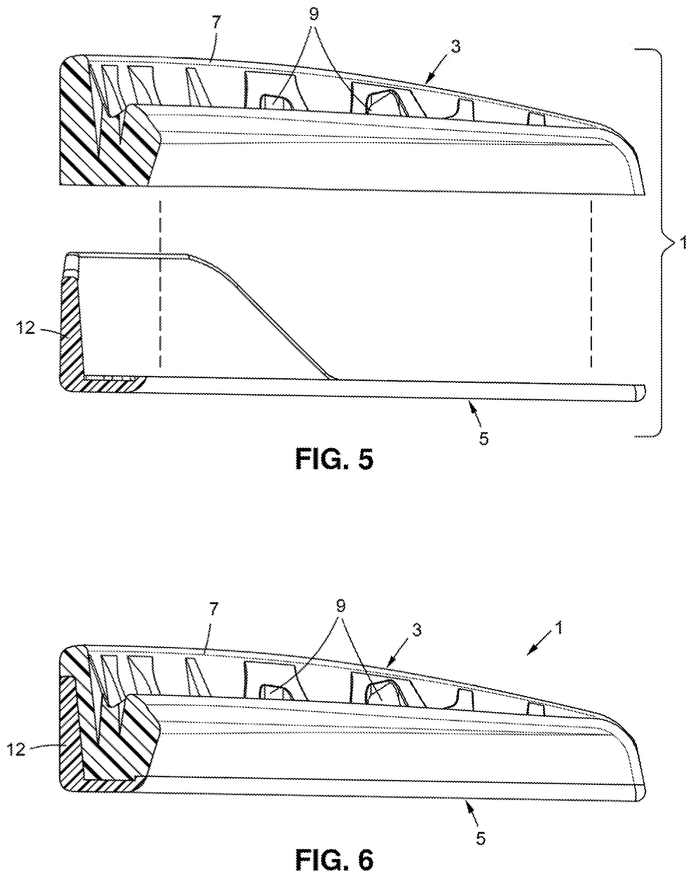

The relatively hard and dense lower chassis 5 of the dental mouth guard 1 against which the upper impression tray 3 is compressed in response to a biting force generated by the user has a segmented bite-resistant pad extending therearound. When the mouth guard 1 is assembled to create a one-piece appliance as shown in FIGS. 5 and 6 and inserted into the user's mouth at night, the protective pad 10 functions as an internal barrier lying between the user's upper and lower sets of teeth. More particularly, the protective pad 10 establishes a raised protective surface that runs continuously around the arcuate lower chassis 5 to prevent the user from inadvertently biting completely through the mouth guard 1 should he clench his teeth tightly together while sleeping after the mouth guard has been placed in his mouth.

The lower chassis 5 also has a thin wall 12 that stands upwardly from the front thereof to lie in front of and be embedded within the upper impression tray 3 to function as a buffer between the mouth guard 1 and the user's (e.g., upper) lip. The upstanding front wall 12 of the lower chassis 5 provides a smooth continuous surface to be positioned against the inside of the user's lip to achieve an advantage that will be described when referring hereinafter to FIG. 7.

FIGS. 5 and 6 of the drawings show the dental mouth guard 1 during its one-piece assembly after which it is ready to be customized for use in the mouth of an individual seeking to prevent bruxism while sleeping. The integral assembly of the mouth guard 1 is completed when the upper impression tray 3 is positioned on top of and molded to the lower chassis 5 so that the impression tray and chassis lay one above the other. Accordingly, the upper impression tray 3 is seated on top of the protective pad 10 of the lower chassis 5 with the set of open holes 9 molded into the upper impression tray 3 lying over the pad 10 and facing upwardly at which to be responsive to a biting force imparted thereto by the user's (e.g., upper) set of teeth.

Turning now to FIG. 7 of the drawings, the assembled one-piece dental mouth guard 1 is shown being removably received by a single use, disposable fitting tray 20. The fitting tray 20 has an arcuate (i.e., arch) shaped submersible channel 22 at one end thereof which is curved to match the arch of the mouth guard 1. The submersible channel 22 has front and rear walls 24 and 26 that are spaced from one another to enable the fitting tray 20 to receive therewithin and transport the mouth guard 1. The channel also has a closed bottom 27 located between the front and rear walls 24 and 26 upon which the mouth guard 1 is seated. Lying opposite and extending from the channel 22 of the fitting tray 20 is an elongated handle 28 to be grasped in the hand of the user. The handle 28 has a plurality of optional holes 30 formed therethrough so as to enable the handle 28 to be more reliably gripped in the hand of the user should it become wet.

A pair of continuously extending vertical and horizontal adjustment slots 32 and 34 are formed through the front wall 24 and the closed bottom 27 of the submersible channel 22 of the fitting tray 20. The adjustment slots 32 and 34 advantageously allow the channel 22 to flex and expand and thereby compensate for the user having an especially wide or narrow jaw by which a correspondingly variable biting force will be applied against the mouth guard 1 being carried by the impression setting tray 20.

FIG. 7 also shows the fitting tray 20 and the one-piece mouth guard 1 that is transported therewithin being located in a container filled with boiling water. The user holds or leans the handle 28 so that the submersible channel 22 of the fitting tray 20 is located within the boiling water for about one minute in order for the relatively soft upper impression tray 3 of the mouth guard 1 to be heated and softened. The impression setting tray 20 is then removed from the boiling water by means of the handle 28, and the mouth guard 1 is allowed to momentarily cool. The fitting tray 20 is now inserted into the user's mouth so that the upper impression tray 3 of the mouth guard 1 will surround the user's (e.g., upper) set of teeth.

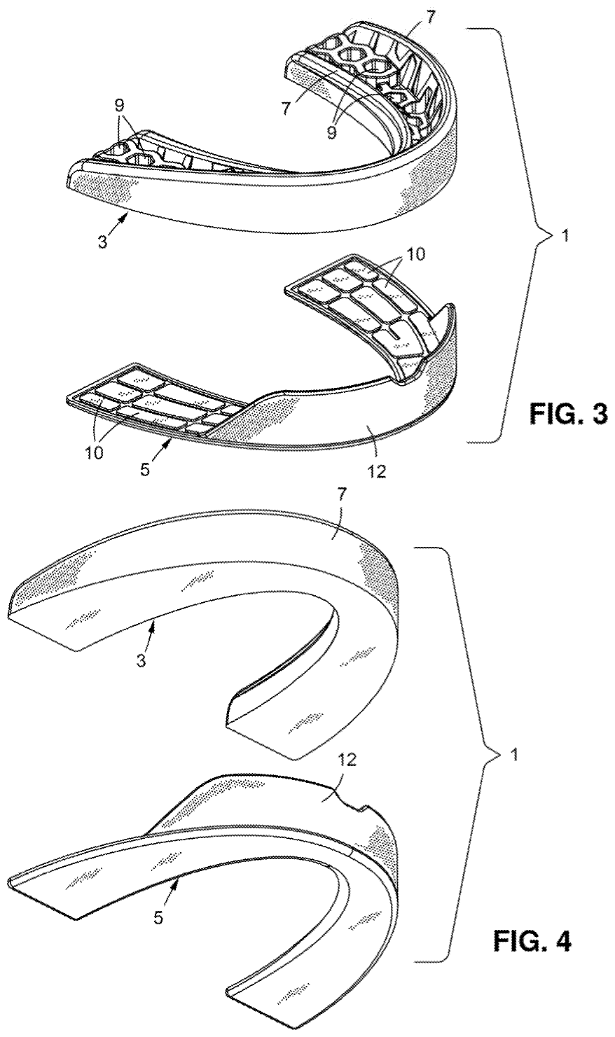

The user then closes his mouth and bites down on the mouth guard 1 and into the heated and softened upper impression tray 3 thereof. Accordingly, an accurate impression of the user's (upper) teeth will be made in the upper impression tray. As was previously explained, the raised protective pads 10 shown in FIGS. 2 and 3 prevent the user from biting through the lower chassis 5 of the mouth guard 1. As was also explained, because the lower chassis 5 is harder than the upper impression tray 3, the biting force generated by the user's jaws will cause the upper impression tray 3 to be compressed against the lower chassis 5 so that the shape of the user's (upper) set of teeth is reliably impressed into the upper impression tray. At the same time, the buffer wall 12 standing upwardly from the relatively hard lower chassis 5 to lie in front of the upper impression tray 3 will prevent the softened material of the upper impression tray 3 from migrating under compression through the pair of adjustment slots 32 and 34 that are formed in the channel 22 of the fitting tray 20. The upstanding buffer wall 12 provides the advantage of preventing the formation of bumps around the mouth guard 1 that might irritate the user's lip should he continuously open and close his mouth while sleeping with the mouth guard lying over his (e.g., upper) set of teeth.

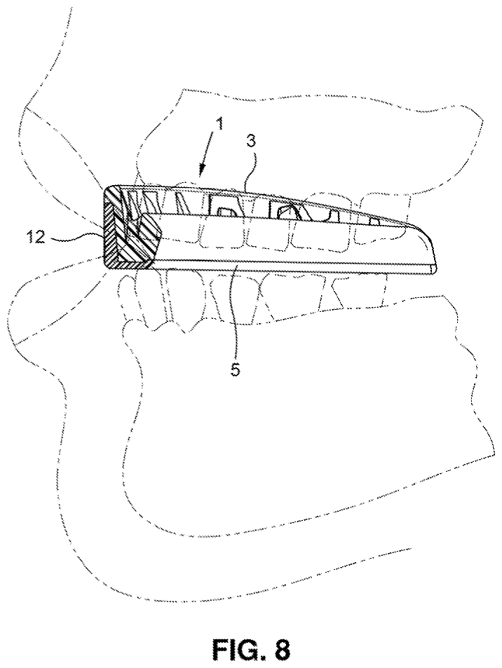

Next, the handle 28 of the fitting tray 20 is once again gripped, and the mouth guard 1 is withdrawn from the user's mouth. The mouth guard 1 is then cooled in cold water to cause the thermal impression of the user's teeth to set. The fitting tray 20 is now separated from the mouth guard 1 and discarded. The mouth guard 1 having the shape of the user's (upper) set of teeth impressed therein is now ready to be placed in the user's mouth at night to surround his (upper) set of teeth in the manner shown at FIG. 8 of the drawings to thereby prevent bruxism and the grinding of his upper and lower sets of teeth against one another while the user sleeps through the night. Because of the accurate and close fit that will be made by the dental mouth guard 1 of this invention over and around the user's teeth, the mouth guard is unlikely to shift its position in the user's mouth and move off his teeth as the user changes his position in bed.

1. A combination comprising:
a dental appliance being configured to fit in the mouth and over at least one of the upper and lower sets of teeth of an individual during sleep to prevent bruxism and the upper and lower sets of teeth of the individual from grinding together, said dental appliance having an upper impression tray to receive a biting force applied thereto after said dental appliance is heated and a lower chassis to which said upper impression tray is attached; and
a fitting tray within which said dental appliance is removably received and transported, said fitting tray having a handle at which said fitting tray is grasped and carried and said dental appliance is correspondingly transported.

2. The combination recited in claim 1, wherein the upper impression tray of said dental appliance is responsive to heat so as to become soft and receive an impression of the at least one of the individual's upper and lower sets of teeth in response to the biting force applied thereto after said dental appliance is heated.

3. The combination recited in claim 2, further comprising a source of heat to heat and soften the upper impression tray of said dental appliance so that the impression of the at least one of the individual's upper and lower sets of teeth can be made therein, said fitting tray transporting said dental appliance to said source of heat to be heated thereby.

4. The combination recited in claim 1, wherein the lower chassis of said dental appliance is harder and denser than the upper impression tray of said dental appliance.

5. The combination recited in claim 1, wherein each of the upper impression tray and the lower chassis of said dental appliance is manufactured from a copolymer material such that said upper impression tray is molded over and attached to said lower chassis.

6. The combination recited in claim 1, wherein the lower chassis of said dental appliance has an arcuate shape and a bite-resistant protective pad extending therearound, the upper impression tray of said dental appliance being seated on said protective pad.

7. The combination recited in claim 1, wherein the upper impression tray of said dental appliance has an arcuate shape and a bite impression channel against which the biting force is applied after said dental appliance is heated, said bite impression channel having a plurality of holes formed therein and extending therearound.

8. The combination recited in claim 1, wherein said fitting tray has an arcuate shaped channel within which said dental appliance is removably recited and transported, the handle of said fitting tray being attached to and extending from said channel.

9. The combination recited in claim 8, wherein the arcuate shaped channel of said fitting tray has at least adjustment slot formed therein by which to permit said arcuate shaped channel to expand in response to the biting force applied to the upper impression tray of said oral appliance received within said arcuate shaped channel.

10. The combination recited in claim 9, wherein the lower chassis of said dental appliance has a buffer wall standing upwardly therefrom and lying in front of the upper impression tray of said dental appliance, said buffer wall blocking said upper impression tray from migrating through the adjustment slot formed in said fitting tray in response to the biting force being applied to said upper impression tray.

11. A dental appliance being configured to fit in the mouth and over at least one of the upper and lower sets of teeth of an individual during sleep to prevent bruxism and the upper and lower sets of teeth of the individual from grinding together, said dental appliance having a heat responsive and arcuate shaped upper impression tray within which the at least one of the individual's upper and lower sets of teeth is received and an arcuate shaped lower chassis to which said upper impression tray is bonded such that the upper impression tray and the lower chassis lie one above the other, said lower chassis being harder and denser than said upper impression tray.

12. The dental appliance recited in claim 11, wherein said lower chassis has a buffer wall standing upwardly therefrom and embedded in the front of said upper impression tray so as to lie between said upper impression tray and the mouth of the individual when said dental appliance is positioned over the at least one of the individual's upper and lower sets of teeth.

13. The dental appliance recited in claim 11, wherein said lower chassis has a plurality of raised pads extending therearound, said upper impression tray being seated on said plurality of raised pads.
